(19) United States
(12) Patent Application Publication
(10) Pub. No.: US 2013/0296331 A1
(43) Pub. Date: Nov. 7, 2013

(54) COMPOSITIONS AND METHODS FOR AMELIORATING RENAL DYSFUNCTION INDUCED BY RENAL HYPOPERFUSION OR ACUTE KIDNEY INJURY

(71) Applicants: Zaid Abassi, Haifa (IL); Bishar Bishara, Haifa (IL)

(72) Inventors: Zaid Abassi, Haifa (IL); Bishar Bishara, Haifa (IL)

(73) Assignee: Technion Research and Development Foundation Ltd., Haifa (IL)

(21) Appl. No.: 13/989,204

(22) PCT Filed: Nov. 24, 2011
(86) PCT No.: PCT/IL2011/000900

§ 371 Date: Jun. 19, 2013

Related U.S. Application Data
(60) Provisional application No. 61/417,366, filed on Nov. 26, 2010.

(51) Int. Cl. A61K 31/4985

(52) U.S. Cl. CPC A61K 31/4985
USPC 514/250

(57) ABSTRACT

The invention provides compositions and methods for ameliorating renal dysfunction induced by renal hypoperfusion or acute kidney injury, resulting for example from increased intra-abdominal pressure, using phosphodiesterase inhibitors, more particularly phosphodiesterase type inhibitors, or pharmaceutically acceptable salts or solvates thereof.

### TECHNICAL FIELD

The present invention relates to compositions and methods for ameliorating renal dysfunction induced by renal hypoperfusion or acute kidney injury resulting, e.g., from increased intra-abdominal pressure, and particularly, to phosphodiesterase inhibitors for use in said compositions and methods.

### BACKGROUND ART

Laparoscopic surgery has the potential to increase the number of living kidney donations by reducing donor complications and morbidity (Demyttenaere et al., 2007; Ratner et al., 1997); however, pneumoperitoneum during laparoscopic surgery has been shown to produce transient oliguria (Chang et al., 1994; Nishio et al., 1999; Richards et al., 1983; Harman et al., 1982) and deterioration of glomerular filtration rate (GFR). Similarly, most of the studies identified a decrease in renal blood flow (RBF) and renal cortical perfusion (Demyttenaere et al., 2007; Chiu et al., 1994; Chiu et al., 1996; Hazebroek et al, 2003; Junghans et al., 1997; Lindberg et al., 2003; London et al., 2000; McDougall et al., 1996). Although the systemic physiologic consequences of increased intra-abdominal pressure (IAP) in general and its adverse effects on renal excretory function and hemodynamics in particular have been extensively studied, the mechanisms underlying the changes in renal physiology during IAP are still not fully understood. It is well known that pneumoperitoneum-induced renal dysfunction is a multi-factorial phenomenon. For instance, the severity of the reduction in renal function following pneumoperitoneum is affected by the level of IAP (Junghans et al., 1997), baseline volume status (London et al., 2000), degree of hypercarbia (Kirsch et al., 1994), positioning (Junghans et al., 1997) and individual hemodynamic and renal reserves. Additional factors which have been proposed as contributing to renal dysfunction during pneumoperitoneum include direct compression of the renal parenchyma and renal vein (Chiu et al., 1994; Ho et al., 1995), increased resistance of renal vasculature (Zacherl et al., 2003), activation of neurohormonal systems including vasopressin (ADH), endothelin-1 (ET-1) (Hamilton et al., 1998; Ambrose et al., 2001), the rennin-angiotensin-aldosterone system (RAAS), catecholamines (Gudmundsson et al., 2003; Joris et al., 1998) and reduction in cardiac output (Ho et al., 1995; Joris et al., 1998). In contrast, compression of the ureter has now been ruled out as a factor contributing to the oliguria (Richards et al., 1983; Harman et al., 1982; McDougall et al., 1996). Most recently, histological effects of IAP such as tubular necrosis and apoptosis, interstitial hemorrhagic in subcapsular area with congestion of the glomerular and peritubular capillaries were implicated in IAP-induced renal injury (Shimizu et al., 2004, 2006; Khoury et al, 2008). The fact that most of these changes are also observed in acute kidney injury (AKI) suggests that IAP provokes acute renal failure.

Although the transient renal dysfunction during laparoscopy has not been shown to result in any permanent effects in the donor (Hazebroek et al., 2003; Nguyen et al., 2002), concerns have been raised that these undesired renal effects may predispose to altered allograft function in the recipient (Nogueira et al., 1999). Likewise, patients who have pre-existing renal dysfunction are at increased risk of renal complications associated with laparoscopic surgery, and in certain cases may require renal replacement therapy, i.e., dialysis (de Seigneux et al., 2011). Experimental evidence has accumulated in recent years indicating that locally produced vasoactive substances such as nitric oxide (NO) play a fundamental role in the regulation of systemic and intra-renal hemodynamics, pressure natriuresis, release of sympathetic neurotransmitters and renin, and tubular solutes and water transport (Demyttenaere et al., 2007; Kone, 2004; Lahera et al., 1991; Mattson et al., 1992; Moncada et al., 1991). However, the involvement of NO system in the adverse effects of pneumoperitoneum on renal perfusion and function has not yet been explored. For this purpose we recently investigated the involvement of the NO in pneumoperitoneum-induced renal dysfunction in rats (Abassi et al., 2008; Bishara et al., 2009). In these studies, we demonstrated that IAP of 14 mm Hg, but not of 7 mm Hg, decreased renal excretory function and hypofiltration in rats and that these effects could be partially ameliorated by pretreatment with nitroglycerine, an NO donor. Concomitant with these findings, pretreatment with nitro-L-arginine methyl ester (L-NAME), an inhibitor of NO synthase, aggravated IAP-induced renal dysfunction. Overall, these findings may indicate that patients with endothelial dysfunction may be more sensitive to the adverse renal hemodynamics and kidney function seen during pneumoperitoneum. Moreover, it is possible that IAP of less than 14 mm Hg may be safe for patients with endothelial dysfunction undergoing laparoscopic surgery.

Some proposals have been suggested to overcome the negative renal effects of pneumoperitoneum, including inhibiting the RAAS, aggressive hydration, using IAP lower than 14 mm Hg (Borba et al., 2005; Lindstrom et al., 2003), and preconditioning consisting of 10 min of pneumoperitoneum followed by 10 min of deflation, which was shown to decrease the oxidative stress in the plasma, liver, kidney and other organs (Yilmaz et al., 2003). Nevertheless, none of these approaches completely abolishes the deleterious consequences of pneumoperitoneum on renal perfusion and function, thus development of new approaches to minimize the common side effects of laparoscopic surgical procedures is appealing.

### SUMMARY OF INVENTION

It has been found, in accordance with the present invention, that pretreatment of rats with either decompensated congestive heart failure (CHF) or myocardial infarction, with the phosphodiesterase type 5 (PDE5) inhibitor tadalafil (Cialis®), a drug indicated for treatment of pulmonary hypertension, erectile dysfunction and CHF, remarkably attenuated the vulnerability to the adverse renal effects of increased intra-abdominal pressure. As further been found, administration of Cialis® to rats prior to induction of a classical ischemic acute kidney injury by renal artery clamping prevented renal injury compared with non-treated animals, as expressed by reduction of urinary excretion of neutrophil gelatinase-associated lipocalcin (NGAL) and kidney injury molecule 1 (KIM-1), two novel biomarkers of renal damage. These findings indicate that phosphodiesterase inhibitors such as Cialis® can be used for ameliorating, i.e., attenuating, renal dysfunction induced by renal hypoperfusion or acute kidney injury, and particularly wherein said renal hypoperfusion or acute kidney injury results from increased intra-abdominal pressure.

In one aspect, the present invention thus relates to a method for ameliorating renal dysfunction induced by renal hypoperfusion or acute kidney injury in an individual in need thereof, said method comprising administering to said individual a therapeutically effective amount of a phosphodiesterase inhibitor or a pharmaceutically acceptable salt or solvate thereof.

In certain embodiments, the renal hypoperfusion or acute kidney injury results from increased intra-abdominal pressure. In a particular such aspect, the present invention thus relates to a method for ameliorating renal dysfunction induced by increased intra-abdominal pressure in an individual in need thereof, said method comprising administering to said individual a therapeutically effective amount of a phosphodiesterase inhibitor or a pharmaceutically acceptable salt or solvate thereof.

In another aspect, the present invention provides a pharmaceutical composition comprising a phosphodiesterase inhibitor or a pharmaceutically acceptable salt or solvate thereof, and a pharmaceutically acceptable carrier, for ameliorating renal dysfunction induced by renal hypoperfusion or acute kidney injury. In a particular such aspect, the invention provides a pharmaceutical composition comprising a phosphodiesterase inhibitor or a pharmaceutically acceptable salt or solvate thereof, and a pharmaceutically acceptable carrier, for ameliorating renal dysfunction induced by increased intra-abdominal pressure.

In a further aspect, the present invention provides a phosphodiesterase inhibitor or a pharmaceutically acceptable salt or solvate thereof for use in ameliorating renal dysfunction induced by renal hypoperfusion or acute kidney injury. In a particular such aspect, the invention provides a phosphodiesterase inhibitor or a pharmaceutically acceptable salt or solvate thereof for use in ameliorating renal dysfunction induced by increased intra-abdominal pressure.

In still another aspect, the present invention relates to use of a phosphodiesterase inhibitor or a pharmaceutically acceptable salt or solvate thereof for the preparation of a pharmaceutical composition for ameliorating renal dysfunction induced by renal hypoperfusion or acute kidney injury. In a particular such aspect, the invention relates to use of a phosphodiesterase inhibitor or a pharmaceutically acceptable salt or solvate thereof for the preparation of a pharmaceutical composition for ameliorating renal dysfunction induced by increased intra-abdominal pressure.

### BRIEF DESCRIPTION OF DRAWINGS

FIG. 1 schematically shows the experimental model used for the induction of congestive heart failure (CHF) in rats.

FIG. 2 shows the experimental model used for the induction of infra-abdominal pressure (IAP), simulating pneumoperitoneum, in rats.

FIGS. 3A-3D show the effects of IAP, measured in mmHg, on glomerular filtration rate (GFR) (3A-3B) and renal plasma flow (RPF) (3C-3D) in normal rats and animals with compensated or decompensated CHF. (*) p<0.05 vs. baseline; (#) p<0.05 vs. control; ($) p<0.05 vs. compensated.

FIGS. 4A-4C show the effects of IAP, measured in mmHg, on urinary flow rate (4A), urinary sodium excretion (4B) and mean arterial pressure (MAP) (4C) in normal rats and animals with either compensated or decompensated CHF. (*) p<0.05 vs. baseline; (#) p<0.05 vs. control; ($) p<0.05 vs. compensated.

FIGS. 5A-5B show the effects of IAP, measured in mmHg, on absolute urinary cGMP excretion (5A) and normalized urinary cGMP/GFR (5B) in normal rats and animals with either compensated or decompensated CHF. (*) p<0.05 vs. baseline; (#) p<0.05 vs. control.

FIGS. 6A-6D show the effects of the phosphodiesterase type 5 (PDE5) inhibitor Cialis® (PDE-I) on the adverse effects of incremental IAP, measured in mmHg, on GFR (6A-6B) and RPF (6C-6D) in rats with decompensated CHF. (*) p<0.05 vs. baseline; (#) p<0.05 vs. control; ($) p<0.05 vs. decompensated.

FIGS. 7A-7C show the effects of Cialis® (PDE-I) on the adverse effects of incremental IAP, measured in mmHg, on urinary flow rate (V, 7A) and urinary sodium excretion (UnaV, 7B) in rats with decompensated CHF. MAP is shown in 7C. (*) p<0.05 vs. baseline; (#) p<0.05 vs. control; ($) p<0.05 vs. decompensated.

FIG. 8 shows the effects of Cialis® (PDE-I) on absolute urinary cGMP excretion in decompensated CHF rats subjected to incremental IAP, measured in mmHg. (*) p<0.05 vs. baseline; (#) p<0.05 vs. control.

FIGS. 9A-9D show the effects of Cialis® (PDE-I) on the adverse effects of incremental IAP, measured in mmHg, on GFR (9A-9B) and RPF (9C-9D) in rats with myocardial infarction (MI). (*) p<0.05 vs. baseline; (#) p<0.05 vs. control; ($) p<0.05 vs. MI.

FIGS. 10A-10C show the effects of Cialis® (PDE-I) on the adverse effects of incremental IAP, measured in mmHg, on urinary flow rate (V, 10A) and urinary sodium excretion (UnaV, 10B) in rats with MI. MAP is shown in 10C. (*) p<0.05 vs. baseline; (#) p<0.05 vs. control; ($) p<0.05 vs. MI.

FIG. 11 shows the effects of Cialis® (PDE-I) on absolute urinary cGMP excretion (UcGMP) in rats with myocardial infarction subjected to incremental IAP, measured in mmHg. (*) p<0.05 vs. baseline.

FIGS. 12A-12B show the effects of measured in mmHg, on absolute urinary excretion of neutrophil gelatinase-associated lipocalcin (NGAL, 12A); and fold of increase in urinary NGAL from baseline in sham controls and rats with compensated and decompensated CHF (12B).

FIGS. 13A-13B show the effects of Cialis® (PDE-I) on urinary excretion of NGAL (13A) and kidney injury molecule 1 (KIM-1, 13B) in experimental model of acute kidney injury (AKI) induced by renal ischemia of 45 min. (*) p<0.05 vs. untreated animals.

### DETAILED DESCRIPTION OF THE INVENTION

As stated above, no efficient therapeutic strategies to manage the deleterious renal effects of increased intra-abdominal pressure (IAP) are currently available. The most acceptable way to relieve pneumoperitoneum-induced renal dysfunction is extensive hydration; however, this approach may bear some risk including congestion and lung edema, especially in the elderly. In addition, experimental approaches relying mainly on pharmacological compounds including angiotensin converting enzyme inhibitors (ACE-Is), angiotensin receptor blockers (ARBs), endothelin (ET) antagonists or NO donors have been suggested; however, besides their adverse effects, these drugs do not abolish pneumoperitoneum-induced renal dysfunction and at under best of circumstances only partially ameliorate it.

As shown in the Examples section hereinafter, while IAP of 7 mmHg has no adverse effects on renal hemodynamics and excretory functions in normal rats as well as in rats with decompensated congestive heart failure (CHF), IAP of 10 or 14 mmHg in both normal rats and animals with decompensated CHF significantly decreases glomerular filtration rate (GFR) and renal plasma flow (RPF) in association with impairment of urine output and sodium excretion, and remarkably decreases urinary cGMP excretion. Interestingly, the adverse renal function/perfusion of IAP of 10 and 14 mmHg are less profound in compensated CHF rats compared with decompensated CHF rats. As further shown, IAP increases in dose dependent manner the excretion of neutrophil gelatinase-associated lipocalcin (NGAL) and kidney injury molecule 1 (KIM-1), two novel biomarkers of renal damage, in both normal rats and much more profoundly in rats with CHF, providing a keen evidence that the adverse effects of IAP on the kidney function are not solely through hemodynamic changes but due to tissue injury too.

The studies described herein further show that pretreatment of decompensated CHF rats as well as rats with myocardial infarction-induced heart failure with the phosphodiesterase type 5 (PDE5) inhibitor Cialis® remarkably attenuates their vulnerability to the adverse effects of increased IAP, indicating that both decompensated CHF subjects as well as subjects suffering from myocardial infarction appear to be vulnerable to the adverse renal effects of pneumoperitoneum during laparoscopic surgery and may benefit from pretreatment with a phosphodiesterase inhibitor. Furthermore, administration of Cialis® to rats prior to induction of a classical ischemic acute kidney injury by renal artery clamping, in an ischemia-reperfusion model, prevents renal injury compared with non-treated animals, as expressed by significant attenuation of urinary excretion of NGAL, and KIM-1.

In one aspect, the present invention relates to a method for ameliorating, i.e., attenuating, renal dysfunction induced by renal hypoperfusion or acute kidney injury (AKI) in an individual in need thereof, said method comprising administering to said individual a therapeutically effective amount of a phosphodiesterase (PDE) inhibitor or a pharmaceutically acceptable salt or solvate thereof.

The term “phosphodiesterase (PDE) inhibitor” as used herein refers to any chemical compound, which blocks one or more of the subtypes of the enzyme phosphodiesterase. PDE inhibitors are either selective or nonselective, wherein selective PDE inhibitors specifically block a particular subtype of the enzyme and are classified into PDE type 1 (PDE1); PDE type 2 (PDE2); PDE type 3 (PDE3); PDE type 4 (PDE4); PDE type 5 (PDE5); PDE type 6 (PDE6); PDE type 7 (PDE7); PDE type 8 (PDE8); PDE type 9 (PDE9); PDE type 10 (PDE10); and PDE type 11 (PDE11) inhibitors, and nonselective PDE inhibitors block more than one subtype of the enzyme although they may have different affinities to each one of said more than one subtypes.

Since some of the PDEs are either cyclic adenosine monophosphate (cAMP) or cyclic guanosine monophosphate (cGMP) selective hydrolases, and others hydrolyse both cAMP and cGMP, the various PDE inhibitors prevent inactivation of the intracellular second messengers cAMP, cGMP or both, by the respective PDE subtype(s), thereby increasing the intracellular level of said messenger(s). The terms “cAMP-selective phosphodiesterase inhibitor” and “cGMP-selective phosphodiesterase inhibitor”, as used herein, refer to PDE inhibitors as defined above, which block one or more of the subtypes of the enzyme phosphodiesterase thus preventing the biodegradation of cAMP and cGMP, respectively, by the respective phosphodiesterase subtype(s).

Non-limiting examples of PDE1 inhibitors include vinpocetine ((3α,16α)-eburnamenine-14-carboxylic acid ethyl ester), a semisynthetic derivative alkaloid of vincamine that leads to increase in the intracellular levels of cGMP.

Non-limiting examples of PDE2 inhibitors include EHNA, i.e, erythro-9-(2-hydroxy-3-nonly)adenine; and anagrelide (6,7-dichloro-1,5-dihydroimidazo (2,1-b)quinazolin-2(3H)-one), which is a potent cAMP-selective PDE inhibitor.

PDE3 inhibitors lead to an increase in the intracellular level of cAMP, although sometimes referred to as cGMP-inhibited phosphodiesterase as well. Examples of PDE3 inhibitors include, without being limited to, aminone (5-amino-3,4′-bipyridin-6(1H)-one), which inhibits the breakdown of both cAMP and cGMP by the PDE3 enzyme; cilostazol (6-[4-(1-cyclohexyl-1H-tetrazol-5-yl)butoxy]-3,4-dihydro-2 (1H)-quinolinone), having a therapeutic focus on increasing cAMP; milrinone (2-methyl-6-oxo-1,6-dihydro-3,4′-bipyridine-5-carbonitrile), which potentiates the effect of cAMP; and enoximone (4-methyl-5-{[4-(methylsulfanyl)phenyl]carbonyl}-2,3-dihydro-1H-imidazol-2-one).

PDE4 hydrolyzes cAMP to inactive AMP and thus lead to an increase in the intracellular level of cAMP. Examples of PDE4 inhibitors include, without being limited to, mesembrine ((3aS,7aR)-3a-(3,4-dimethoxyphenyl)-1-methyl-2,3,4,5,7,7a-hexahydroindol-6-one); rolipram ((RS)-4-(3-cyclopentyloxy-4-methoxy-phenyl)pyrrolidin-2-one); ibudilast (2-methyl-1-(2-propan-2-ylpyrazolo[1,5-a]pyridin-3-yl) propan-1-one), which acts as a selective PDE4 inhibitor or a non-selective phosphodiesterase inhibitor, depending on the dose; piclamilast (3-(cyclopentyloxy)-N-(3,5-dichloropyridin-4-yl)-4-methoxybenzamide); luteolin (2-(3,4-dihydroxyphenyl)-5,7-dihydroxy-4-chromenone); roflumilast (3-(cyclopropyl methoxy)-N-(3,5-dichloropyridin-4-yl)-4-(difluoromethoxy)benzamide); cilomilast (4-cyano-4-(3-cyclopentyloxy-4-methoxyphenyl)cyclohexane-1-carboxylic acid); and drotaverine ((Z)-1-(3,4-diethoxybenzylidene)-6,7-diethoxy-1,2,3,4-tetrahydro isoquinoline).

PDE5 inhibitors block the degradative action of PDE5 on cGMP, e.g., in the smooth muscle cells lining the blood vessels supplying the corpus cavernosum of the penis. Such drugs are currently used in the treatment of erectile dysfunction, and were the first effective oral treatment available for the condition. Since PDE5 is also present in the arterial wall smooth muscle within the lungs, PDE5 inhibitors have also been explored for the treatment of pulmonary hpertension, a disease in which blood vessels in the lungs become abnormally narrow. Examples of PDE5 inhibitors include, without being limited to, avanafil (4-[(3-chloro-4-methoxybenzyl)amino]-2-[2-(hydroxymethyl)-1-pyrrolidinyl]-N-(2-pyrimidinyl methyl)-5-pyrimidinecarboxamide); lodenafil (bis-(2-{4-[4-ethoxy-3-(1-methyl-7-oxo-3-propyl-6,7-dihydro-1H-pyrazolo[4,3-d]pyrimidin-5-yl)-benzenesulfonyl]piperazin-1-yl}-ethyl)carbonate); mirodenafil (5-ethyl-3,5-dihydro-2-[5-([4-(2-hydroxyethyl)-1-piperazinyl]sulfonyl)-2-propoxyphenyl]-7-propyl-4H-pyrrolo[3,2-d]pyrimidin-4-one); sildenafil (Viagra®; 1-[4-ethoxy-3-(6,7-dihydro-1-methyl-7-oxo-3-propyl-1H-pyrazolo[4,3-d]pyrimidin-5-yl)phenylsulfonyl]-4-methyl piperazine); tadalafil (Cialis®; (6R-trans)-6-(1,3-benzodioxol-5-yl)-2,3,6,7,12,12a-hexahydro-2-methyl-pyrazino[1′,2′:1,6]pyrido[3,4-b]indole-1,4-dione); vardenafil (Levitra®; 4-[2-ethoxy-5-(4-ethylpiperazin-1-yl)sulfonyl-phenyl]-9-methyl-7-propy-3,5,6,8-tetrazabicyclo[4.3.0]nona-3,7,9-trien-2-one); udenafil (Zydena®; 3-(1-methyl-7-oxo-3-propyl-4,7-dihydro-1H-pyrazolo[4,3-d]pyrimidin-5-yl)-N-[2-(1-methylpyrrolidin-2-yl)ethyl]-4-propoxybenzenesulfonamide); dipyridamole (2,2′,2″,2′″-(4,8-di(piperidin-1-yl) pyrimido[5,4-d]pyrimidine-2,6-diyl)bis(azanetriyl)tetraethanol); zaprinast (2-(2-propyloxyphenyl)-8-azapurin-6-one); icariin (5-hydroxy-2-(4-methoxyphenyl)-8-(3-methylbut-2-enyl)-7-[(2S,3R,4S,5S,6R)-3,4,5-trihydroxy-6-(hydroxymethyl)oxan-2-yl]oxy-3-[(2S,3R,4R,5R,6S)-3,4,5-trihydroxy-6-methyloxan-2-yl]oxychromen-4-one); and a methoxyquinazoline such as 6-acetoxy-4-chloro-7-methoxyquinazoline and 2,4-dichloro-7-methoxy quinazoline.

In certain embodiments, the PDE inhibitor used according to the method of the present invention is a cGMP-selective PDE inhibitor. Particular cGMP-selective PDE inhibitors that can be used according to the method of the invention are any of the cGMP-selective PDE inhibitors described above, such as the PDE1 inhibitor vinpocetine, certain PDE3 inhibitors capable of inhibiting the breakdown of both cAMP and cGMP, and any one of the PDE5 inhibitors listed above, as well as any chemical derivative thereof capable of selectively blocking one or more of the subtypes of the enzyme PDE thus preventing the inactivation of cGMP by the respective PDE subtype(s). In more particular embodiments, the cGMP-selective PDE inhibitor used according to the method of the invention is a PDE5 inhibitor such as avanafil, lodenafil, mirodenafil, sildenafil, tadalafil, vardenafil, udenafil, dipyridamole, zaprinast, icariin, a methoxyquinazoline, and any chemical derivative thereof. In a particular specific embodiment, the cGMP-selective PDE inhibitor used according to the method of the invention is tadalafil. Additional cGMP-selective PDE inhibitors that can be used according to the method of the invention are any of the PDE6, PDE7, PDE8, PDE9, PDE10 and PDE11 inhibitors, capable of preventing inactivation of the intracellular second messenger cGMP.

The term “renal hypoperfusion”, as used herein, refers to decreased renal blood flow, which may result from various systemic or local medical conditions such as hypovolemic-, cardiogenic- or septic-shock, renal artery stenosis, renal artery vasoconstriction; administration of vasoconstrictive drugs; exposure to nephrotoxins including exogenous toxins such as contrast agents and aminoglycosides as well as endogenous toxin such as myoglobin; or a renovascular injury, and consequently compromise renal function and lead to prerenal acute renal failure or chronic renal failure.

The term “acute kidney injury” (AKI), as used herein, formerly known as “acute renal failure” (ARF), refers to a rapid loss of kidney function, characterized by particular laboratory findings such as elevated blood urea nitrogen (BUN) and creatinine, or inability of the kidney to produce sufficient amount of urine, i.e., oliguria. The causes for AKI are commonly categorized into prerenal, intrinsic, and postrenal. Prerenal causes of AKI are those that decrease effective blood flow to the kidney, and include systemic causes such as low blood volume, i.e., hypovolemia, low blood pressure, and heart failure, as well as local changes to the blood vessels supplying the kidney such as renal artery stenosis, i.e., a narrowing of the renal artery that supplies the kidney, and renal vein thrombosis, i.e., the formation of a blood clot in the renal vein that drains blood from the kidney. Intrinsic AKI can be due to damage to the glomeruli, renal tubules, or interstitium, which may be caused by glomerulonephritis, acute tubular necrosis (ATN), acute interstitial nephritis (AIN), and nephrotoxic drugs, respectively. Postrenal AKI is a consequence of urinary tract obstruction, which may be related to various conditions such as benign prostatic hyperplasia, kidney stones, obstructed urinary catheter, bladder stone, and bladder-, ureteral- or renal malignancy.

The term “ameliorating renal dysfunction induced by renal hypoperfusion or acute kidney injury”, means attenuating the vulnerability of a kidney to renal hypoperfusion or acute kidney injury leading, inter alia, to decline in GFR and RPF; reduction in both urinary flow rate (V) and urinary sodium excretion (UNaV); reduction in urinary cGMP excretion; and enhanced urinary excretion of neutrophil gelatinase-associated lipocalcin (NGAL) and kidney injury molecule 1 (KIM-1), regardless the cause for said renal hypoperfusion or acute kidney injury, thus improving the functioning of said kidney and attenuating the development of renal failure and renal injury. The term “therapeutically effective amount” as used herein refers to the quantity of the PDE inhibitor or pharmaceutically acceptable salt or solvate thereof, which is useful to ameliorate renal dysfunction, induced by renal hypoperfusion or acute kidney injury, as defined herein.

In certain embodiments, the method of the present invention is aimed at ameliorating renal dysfunction induced by renal hypoperfusion or acute kidney injury, wherein said renal renal hypoperfusion or acute kidney injury results from increased intra-abdominal pressure (IAP).

In fact, in a particular aspect, the present invention relates to a method for ameliorating, i.e., attenuating, renal dysfunction induced by increased IAP in an individual in need thereof, said method comprising administering to said individual a therapeutically effective amount of a PDE inhibitor or a pharmaceutically acceptable salt or solvate thereof.

IAP is the steady-state pressure concealed within the abdominal cavity. IAP increases with inspiration and decreases with expiration, and it is directly affected by the volume of the solid organs or hollow viscera, which may be either empty or filled with air, liquid or fecal matter; the presence of ascites, blood or other space-occupying lesions such as tumors or a gravid uterus; and the presence of conditions that limit expansion of the abdominal wall, such as burn eschars or third-space edema. Increased IAP, also referred to as intra-abdominal hypertension (IAH), is associated with various clinical conditions and occurs frequently in patients with acute abdominal syndromes such as, without being limited to, ileus, intestinal perforation, peritonitis, acute pancreatitis and trauma. An elevated IAP may lead to IAH and abdominal compartment syndrome, which are both etiologically related to an increased morbidity and mortality of critically ill patients. The normal value of IAP is around 2 mmHg, wherein a value above 15 mmHg is considered intra-abdominal hypertension and a value above 25 mmHg is considered an indicator of abdominal compartment syndrome that leads to organ failure. As stated above, IAP may further be a result of pneumoperitoneum, i.e., air or gas in the abdominal (peritoneal) cavity, deliberately created by the surgical team by insufflating the abdomen with carbon dioxide in order to perform laparoscopic surgery.

In particular embodiments, the increased IAP is caused by a laparoscopic surgery. In certain particular embodiments, the PDE inhibitor or the pharmaceutically acceptable salt or solvate thereof used according to the method of the invention is administered prior to, during, or after said laparoscopic surgery. In more particular embodiments such as those exemplified in the Examples section, the PDE inhibitor or the pharmaceutically acceptable salt or solvate thereof used is administered prior to the laparoscopic surgery.

In other particular embodiments, the increased IAP is caused by ascites. In certain particular embodiments, the ascites resulting in increased IAP is caused by cirrhosis or CHF. In other certain particular embodiments, the individual treated according to the method of the invention is a peritoneal dialysis-treated individual having an end-stage renal disease (ESRD), also known as stage 5 kidney disease, signifying the final stage of kidney disease, when actual kidney failure occurs.

In certain embodiments, the method of the present invention is aimed at ameliorating renal dysfunction induced by renal hypoperfusion or acute kidney injury, wherein said acute kidney injury results from renal ischemic insult.

The term “renal ischemic insult”, as used herein, refers to a medical condition resulting from a generalized or localized impairment of oxygen and nutrient delivery to, and waste product removal from, cells of the kidney, leading to mismatch of local tissue oxygen supply and demand, and accumulation of waste products of metabolism. As a result of this imbalance, the tubular epithelial cells undergo injury and, if it is severe, death by apoptosis and necrosis (acute tubular necrosis; ATN), with organ functional impairment of water and electrolyte homeostasis and reduced excretion of waste products of metabolism.

In particular embodiments, the renal ischemic insult is caused by endothelial dysfunction. In certain particular embodiments, the endothelial dysfunction is associated with a disease, disorder or condition such as heart failure, more particularly CHF, myocardial ischemia or myocardial infarction (MI), diabetes, hypertension, hyperlipidemia, atherosclerosis, obesity and renal failure; or with smoking. In other certain particular embodiments, the endothelial dysfunction is associated with smoking or aging.

In certain embodiments, the method of the present invention is aimed at ameliorating renal dysfunction induced by renal hypoperfusion or acute kidney injury, wherein said acute kidney injury is caused by a radiocontrast agent or a nephrotoxic drug.

The term “radiocontrast agent”, as used herein, refers to a type of medical contrast medium used to enhance the contrast of internal bodily structures, e.g., blood vessels and the gastrointestinal tract, in medical imaging thus improve the visibility of said structures in an X-ray based imaging techniques such as computed tomography or radiography commonly known as X-ray imaging. Radiocontrast agents are typically iodine or barium compounds. Commonly used iodinated contrast agents include, without being limited to, high osmolar, i.e., ionic, contrast agents such as diatrizoate (Hypaque 50), metrizoate (Isopaque 370) and iozaglate (Hexabrix), and low osmolar, i.e., non-ionic, contrast agents such as iopamidol (Isovue 370), iohexyl (Omnipaque 350), ioxilan (Oxilan 350), iopromide (Ultravist 370) and iodixanol (Visipaque 320). A commonly used barium-based contrast agent is barium sulfate, mainly used in the imaging of the gastrointestinal tract.

The terms “nephrotoxic drug” and “nephrotoxic agent”, used herein interchangeably, refer to a drug or agent displaying nephrotoxicity, i.e., a poisonous effect on the kidneys. Examples of drugs that can be toxic to the kidney include, without being limited to, antibiotics, primarily aminoglycosides, sulphonamides, amphotericin B, polymyxin, neomycin, bacitracin, rifampin, trimethoprim, cephaloridine, methicillin, aminosalicylic acid, oxy- and chlorotetracyclines; analgesics such as acetaminophen (Tylenol); nonsteroidal anti-inflammatory drugs, e.g., aspirin and ibuprofen; prostaglandin synthetase inhibitors; anti-cancer drugs such as cyclosporin, cisplatin, avastain, rituximab and cyclophosphamide; and methemoglobin-producing agents. Additional agents that can be toxic to the kidney include, without limiting, heavy metals such as lead, mercury, arsenic and uranium; solvents and fuels, such as carbon tetrachloride, methanol, amyl alcohol and ethylene glycol; and herbicides and pesticides.

In another aspect, the present invention provides a pharmaceutical composition comprising a PDE inhibitor as defined above or a pharmaceutically acceptable salt or solvate thereof, and a pharmaceutically acceptable carrier, for ameliorating, i.e., attenuating, renal dysfunction induced by renal hypoperfusion or acute kidney injury. In a particular such aspect, the pharmaceutical composition of the invention is used for ameliorating renal dysfunction induced by increased IAP.

In certain embodiments, the PDE inhibitor comprised within the pharmaceutical composition of the present invention is a cGMP-selective PDE inhibitor as defined above. In particular embodiments, said cGMP-selective PDE inhibitor is a PDE type 5 (PDE5) inhibitor such as avanafil, lodenafil, mirodenafil, sildenafil, tadalafil, vardenafil, udenafil, dipyridamole, zaprinast, icariin, and a methoxyquinazoline. In a more particular embodiment, said PDE5 inhibitor is tadalafil.

The pharmaceutical compositions of the present invention can be provided in a variety of formulations and dosages, and are useful for ameliorating renal dysfunction induced by either renal hypoperfusion or acute kidney injury, regardless the cause for said renal hypoperfusion or acute renal injury. In certain embodiments, the renal hypoperfusion or acute renal injury results from increased IAP that may be caused, e.g., by a laparoscopic surgery, or ascites such as that resulting from cirrhosis or CHF. In other embodiments, the acute kidney injury results from renal ischemic insult such as that caused by endothelial dysfunction associated, e.g., with a disease, disorder or condition such as heart failure, more particularly CHF, myocardial ischemia or myocardial infarction, diabetes, hypertension, hyperlipidemia, atherosclerosis, obesity or renal failure; or with smoking or aging. In further embodiments, the acute kidney injury is caused by a radiocontrast agent or a nephrotoxic drug.

In one embodiment, the pharmaceutical composition of the invention comprises a non-toxic pharmaceutically acceptable salt or solvate of a PDE inhibitor as defined above. Suitable pharmaceutically acceptable salts include acid addition salts such as, without being limited to, the hydrochloride salt, the hydrobromide salt, the mesylate salt; the esylate salt; the benzoate salt, the sulfate salt, the fumarate salt, the p-toluenesulfonate salt, the maleate salt, the succinate salt, the acetate salt, the citrate salt, the tartrate salt, the carbonate salt, and the phosphate salt. Additional pharmaceutically acceptable salts include salts of ammonium (NH₄⁺) or an organic cation derived from an amine of the formula R₄N⁺, wherein each one of the Rs independently is selected from H, C₁-C₂₂, preferably C₁-C₆ alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine. Furthermore, where the PDE inhibitors used according to the invention carry an acidic moiety, suitable pharmaceutically acceptable salts thereof may include metal salts such as alkali metal salts, e.g., lithium, sodium or potassium salts, and alkaline earth metal salts, e.g., calcium or magnesium salts.

Pharmaceutically acceptable salts of PDE inhibitors used according to the present invention may be formed by conventional means, e.g., by reacting a free base form of the active agent, i.e., the PDE inhibitor or the pharmaceutically acceptable salt or solvate thereof, with one or more equivalents of the appropriate acid in a solvent or medium in which the salt is insoluble, or in a solvent such as water which is removed in vacuo or by freeze drying, or by exchanging the anion/cation on a suitable ion exchange resin.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent or ingredient, i.e., the PDE inhibitor or the pharmaceutically acceptable salt or solvate thereof used, into association with a liquid carrier, a finely divided solid carrier, or both, and then, if necessary, shaping the product into the desired formulation. The compositions may be in solid, semisolid or liquid form and may further include pharmaceutically acceptable fillers, carriers, diluents or adjuvants, and other inert ingredients and excipients. The compositions can be formulated for any suitable route of administration, e.g., oral, nasogastric, nasoenteric, orogastric, parenteral (e.g., intramuscular, subcutaneous, intraperitoneal, intravenous, intraarterial or subcutaneous injection, or implant), gavage, buccal, nasal, sublingual or topical administration, as well as for inhalation. The dosage will depend on the state of the patient, and will be determined as deemed appropriate by the practitioner.

The pharmaceutical composition of the present invention may be in a form suitable for oral use, e.g., as tablets, troches, lozenges, aqueous, or oily suspensions, dispersible powders or granules, emulsions, hard or soft capsules, or syrups or elixirs. Compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

The pharmaceutical composition of the present invention may be in the form of a sterile injectable aqueous or oleaginous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

The pharmaceutical compositions of the invention may be in any suitable form, e.g., tablets, such as matrix tablets, in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity.

The pharmaceutical compositions of the invention may comprise the active agent formulated for controlled release in microencapsulated dosage form, in which small droplets of the active agent are surrounded by a coating or a membrane to form particles in the range of a few micrometers to a few millimeters, or in controlled-release matrix.

Another contemplated formulation is depot systems, based on biodegradable polymers, wherein as the polymer degrades, the active ingredient is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions according to the present invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

In a further aspect, the present invention provides a PDE inhibitor as defined above, or a pharmaceutically acceptable salt or solvate thereof, for use in ameliorating, i.e., attenuating, renal dysfunction induced by renal hypoperfusion or acute kidney injury. In a particular such aspect, the PDE inhibitor or pharmaceutically acceptable salt or solvate thereof is used in ameliorating renal dysfunction induced by increased IAP.

In still another aspect, the present invention relates to use of a PDE inhibitor as defined above, or a pharmaceutically acceptable salt or solvate thereof, for the preparation of a pharmaceutical composition for ameliorating, i.e., attenuating, renal dysfunction induced by renal hypoperfusion or acute kidney injury. In a particular such aspect, the PDE inhibitor or pharmaceutically acceptable salt or solvate thereof is used for the preparation of a pharmaceutical composition for ameliorating renal dysfunction induced by increased IAP.

The invention will now be illustrated by the following non-limiting Examples.

### EXAMPLES

### Experimental

Experimental Model of Congestive Heart Failure (CHF):

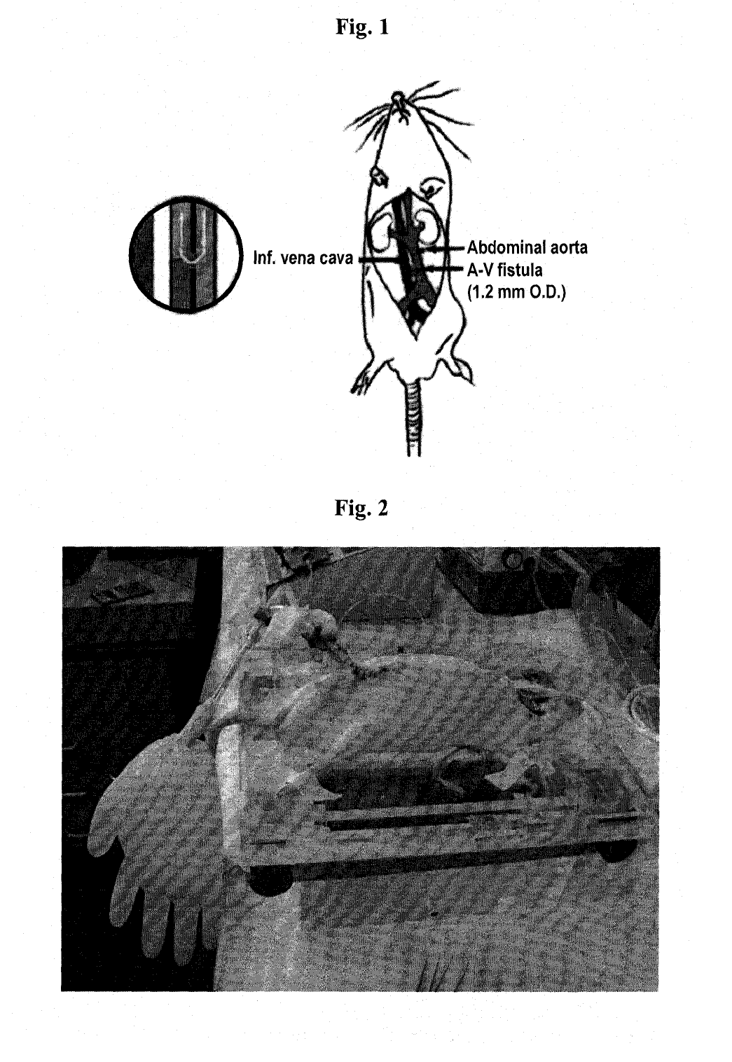

CHF was induced by surgical creation of aorto-caval fistula (ACF) between the abdominal aorta and the vena cava, as schematically shown in FIG. 1. Rats with ACF display two patterns: compensated CHF rats which exhibit normal sodium excretion, and decompensated CHF rats which develop severe sodium retention and die within 7-10 days. Similar pattern was reported in clinical CHF.

Experimental Model of Myocardial Infarction (MI):

Rats were anesthetized with a combination of ketamine (87 mg/kg) and xylazine (13 mg/kg), intubated, and mechanically ventilated at a rate of 80-90 cycles per minute with a tidal volume of 1-2 ml/100 gr. MI was induced by the most common method. Specifically, the left anterior descending (LAD) artery was completely and permanently occluded. Consequently, a large area of the left ventricle wall was deprived of its blood supply, and was thus exposed to hypoxia, ischemia, nutrient starvation and other conditions that develop under these circumstances. Seven days after LAD inclusion, the rats were used in the experimental model of pneumoperitoneum described below.

Experimental Model of Pneumoperitoneum:

The abdomen was opened via small incision in the lower third between the xiphoid and pubis and the urinary bladder was catheterized for urine collection. A Veress needle was inserted into the abdominal cavity and connected to the CO₂ gas supply to maintain intra-abdominal pressure (IAP) at the desired level using a special insufflator (Aesculap, Tuttlingen, Germany). The muscle and skin layers of the abdominal wall were closed separately by silk sutures in an airtight manner (FIG. 2).

Experimental Model of Renal Ischemia:

Male Sprague Dawley rats weighing 300-350 gr were housed under standardized conditions for 2-3 days. Following an overnight fast, the animals were anaesthetized with an inactin anesthesia (100 mg/kg, IP) and placed on a controlled heating (thermoregulated) table keeping the body temperature at 37° C. Polyethylene tubes (PE50) were inserted into the right carotid artery for blood pressure monitoring and blood sampling, and into the jugular vein for infusion of 0.9% normal saline (0.9% NaCl) at a 1.5 ml/h rate, as well as 2% inulin and 1% para-aminohippuric acid (PAH) at a rate of 1.5 ml/h, by syringe pumps. Arterial blood pressure was continuously monitored with a pressure transducer connected to the carotid arterial line. Additional two catheters were inserted after a supravesical incision into the bladder for urine collection. In order to minimize dehydration, the abdominal area was covered with saline-soaked gauze. After 2 baseline urine collections, the left renal artery and vein were dissected and the perirenal fat was preserved. At the end of the ischemic period, the abdominal cavity was reentered, the clamp was removed and reperfusion was started. Four additional urine samples from the ischemic and non-ischemic control kidney were collected each for 60 min for 4 hours. To minimize dehydration of the exposed tissues, the abdominal area was covered with saline-soaked gauze. Urinary samples were analyzed for neutrophil gelatinase-associated lipocalcin (NGAL) and kidney injury molecule 1 (KIM-1), two novel biomarkers of renal damage.

Experimental Protocols:

Male Sprague-Dawley rats were organized into several groups. Normal rats, rats with compensated or decompensated CHF (n=12-13), and rats with MI (n=7) were subjected to IAP of 0 (baseline), 7, 10 or 14 mmHg, over 45-60 min for each pressure, followed by deflation period of 60 min (recovery), as illustrated in Scheme 1. Six additional rats with decompensated CHF and seven additional rats with MI were pretreated with the phosphodiesterase type 5 (PDE5) inhibitor Cialis® (tadalafil; 10 mg/kg/day) for 4 days prior to the experiment. Urine flow rate (V), absolute Na⁺ excretion (UNaV), glomerular filtration rate (GFR), renal plasma flow (RPF) and blood pressure were determined throughout the experiments.

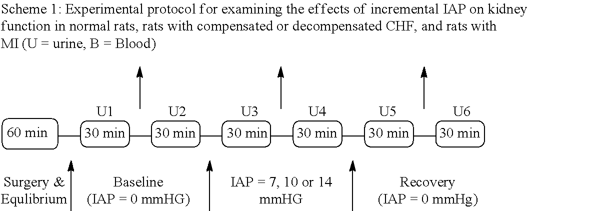

### Example 1

### The Effects of Pneumoperitoneum on GFR and RPF in Normal Rats and Animals with Compensated or Decompensated CHF

In this study, the effects of incremental IAP, simulating pneumoperitoneum (air or gas in the abdominal cavity) and deliberately created in order to perform laparoscopic surgery, on glomerular filtration rate (GFR) and renal plasma flow (RPF) in normal rats and animals with compensated or decompensated CHF were tested.

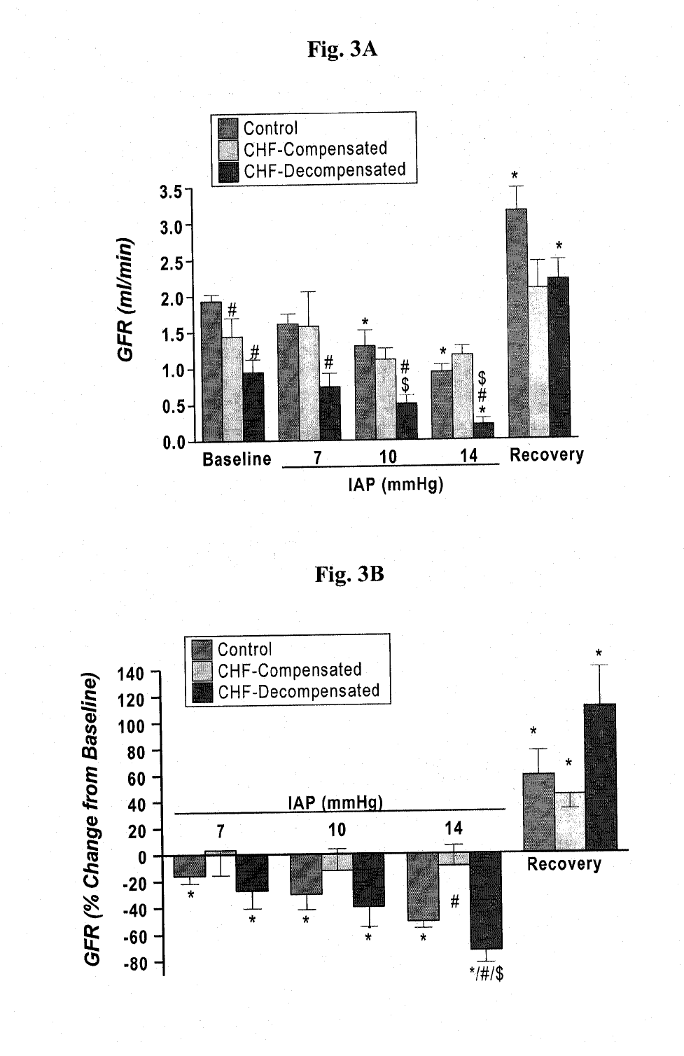

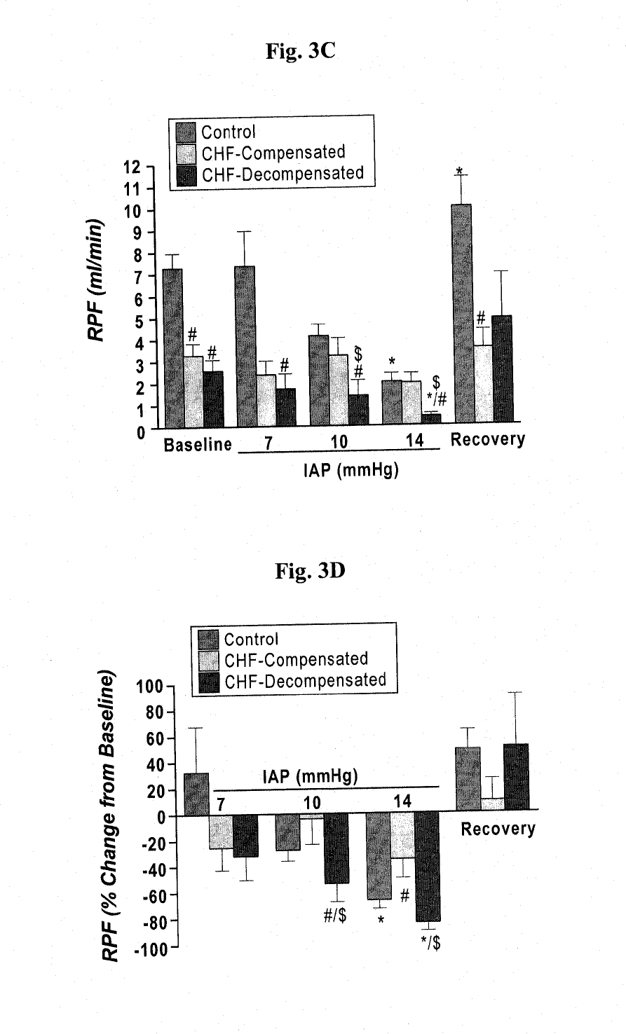

As shown in FIGS. 3A-3D, normal rats subjected to increased IAP showed a decline in GFR (3A) and RPF (3C) as a function of the IAP magnitude, wherein the most adverse renal effects were noticed following the highest IAP tested, i.e., 14 mmHg. Interestingly, when rats with CHF were subdivided into compensated and decompensated, distinct behavior in response to the adverse renal effects of pneumoperitoneum was noticed. First and as expected, rats with CHF have lower basal GFR and RPF compared with control animals, in correlation with the severity of CHF. Second, while rats with compensated CHF did not show significant adverse renal response to the increased IAP, animals with decompensated CHF displayed a severe decline in GFR and RPF as a function of the IAP magnitude, wherein the most adverse renal effects were noticed following the highest IAP tested, i.e., 14 mmHg. This trend is even more remarkable when the results are expressed as percentage change from baseline (3B and 3D).

### Example 2

### The Effects of Pneumoperitoneum on V and UNaV in Normal Rats and Animals with Compensated or Decompensated CHF

In this study, the effects of incremental IAP, simulating pneumoperitoneum, on urinary flow rate (V), urinary sodium excretion (UNaV) and mean arterial pressure (MAP), in normal rats and animals with compensated or decompensated CHF were tested.

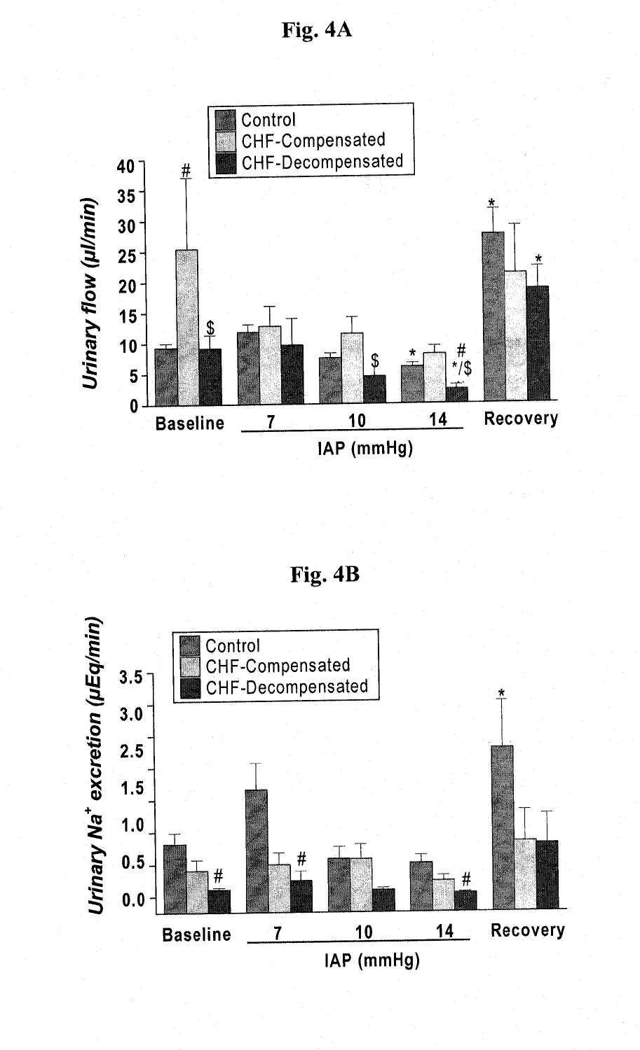

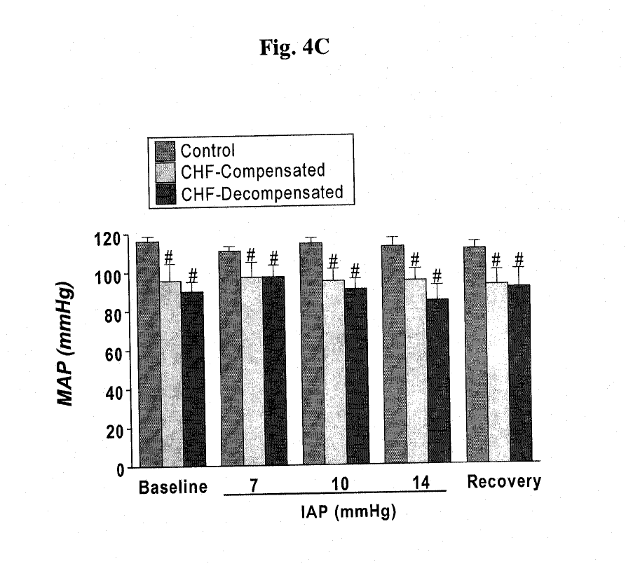

As shown in FIGS. 4A-4C, increasing the IAP caused a significant reduction in both V and UNaV in both controls and decompensated CHF rats, and to a lesser extent in compensated rats, wherein the most adverse renal effects were noticed following the highest IAP tested, i.e., 14 mmHg (4A, 4B). These changes could not be attributed to hypotension effects of the pneumoperitoneum, since the MAP was changed neither in the control rats nor in the CHF animals (4C).

### Example 3

### The Effects of Pneumoperitoneum on Urinary cGMP Excretion in Normal Rats and Animals with Compensated or Decompensated CHF

In this study, the effects of incremental IAP, simulating pneumoperitoneum, on absolute urinary cGMP excretion (UcGMP) and normalized urinary cGMP (UcGMP/GFR) in normal rats and animals with compensated or decompensated CHF were tested.

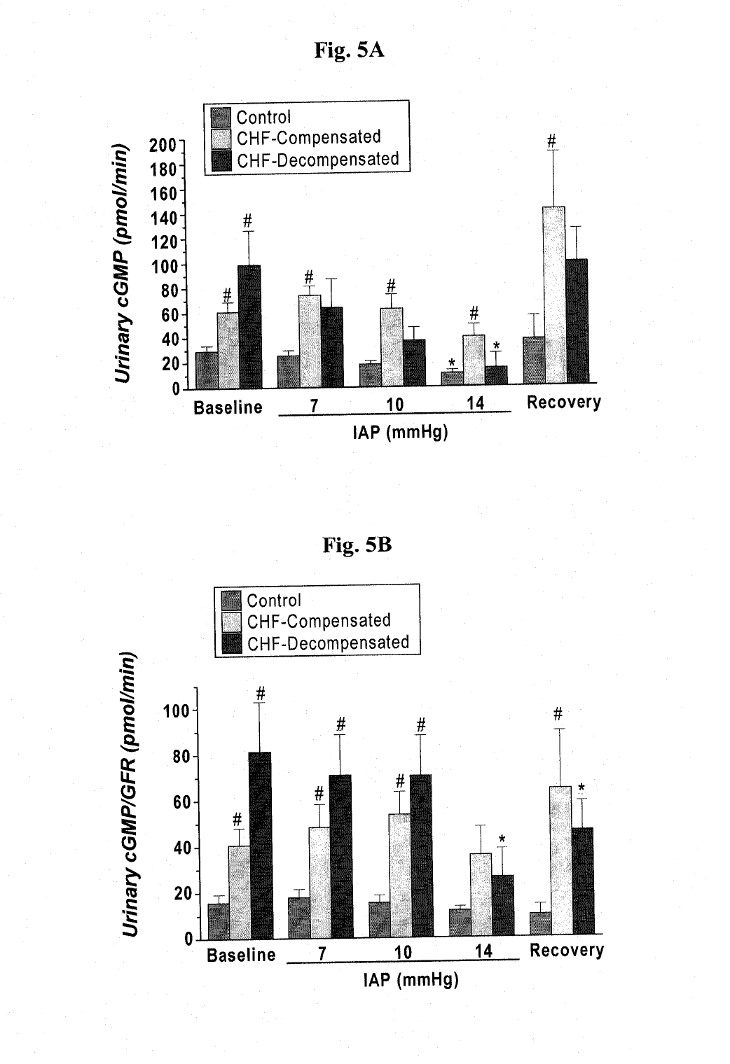

As shown in FIGS. 5A-5B, increasing the IAP caused significant reduction in urinary excretion of cGMP in decompensated rats and to a lesser extent in compensated animals, wherein the most adverse renal effects were noticed following the highest IAP tested, i.e., 14 mmHg (5A). This trend persists even when the UcGMP is normalized to GFR (5B), indicating that there is a correlation between the adverse renal effects of pneumoperitoneum and urinary excretion of cGMP, and that decline in UcGMP may mediate the harmful effects of high IAP.

### Example 4

### PDE5-Inhibitor Remarkably Attenuates the Adverse Effects of Pneumoperitoneum on GFR, RPF, V, UnaV and UcGMP in Rats with Decompensated CHF

In this study, the effects of the phosphodiesterase type 5 (PDE5) inhibitor Cialis® (tadalafil), administered as described in Materials and Methods, on the adverse effects of incremental IAP, simulating pneumoperitoneum, on GFR, RPF, urinary flow rate (V), urinary sodium excretion (UNaV) and urinary cGMP excretion (UcGMP), in rats with decompensated CHF were tested.

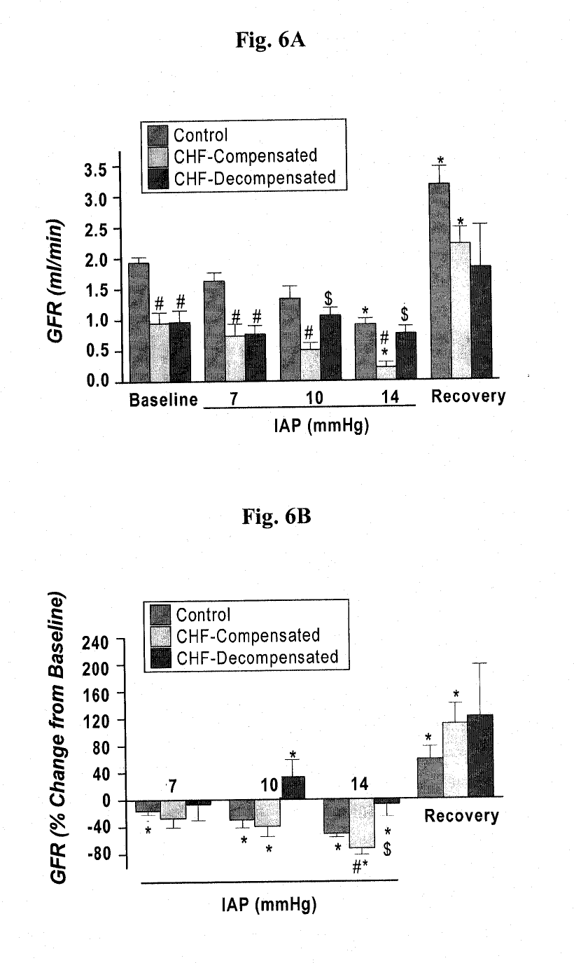

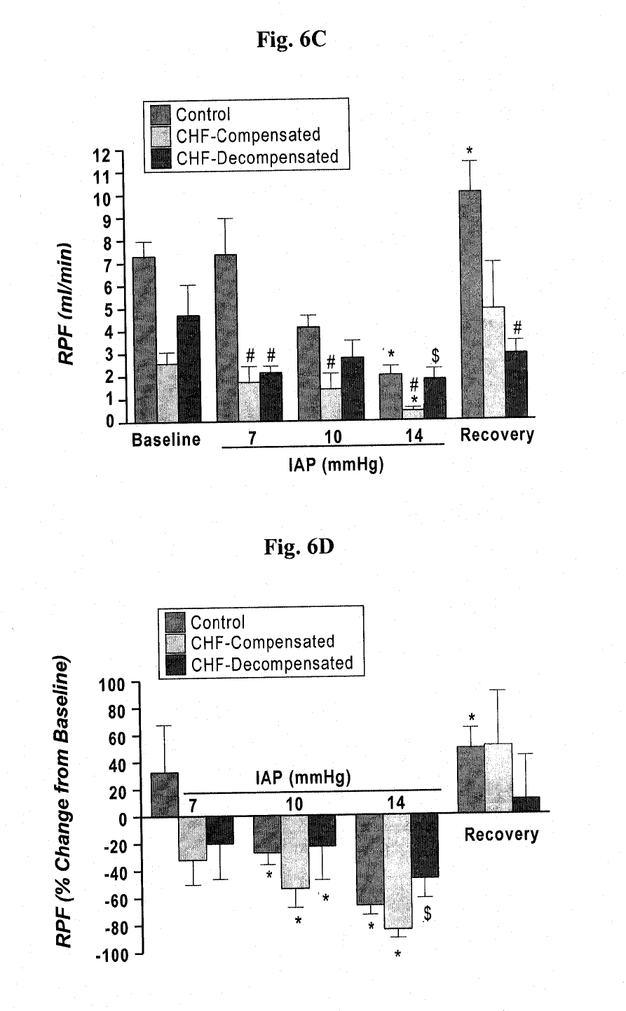

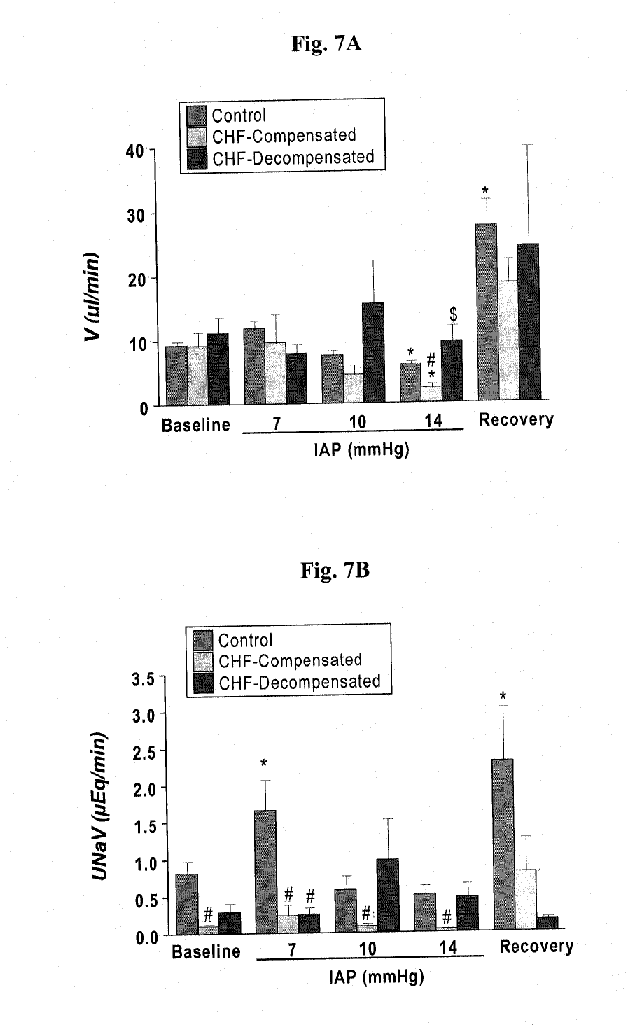

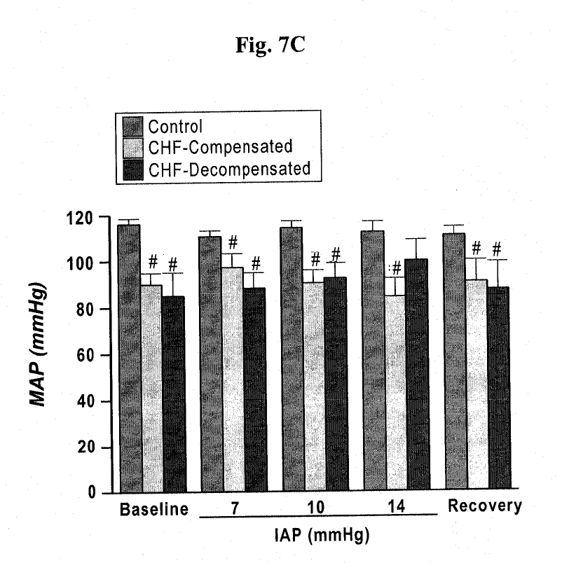

FIGS. 6A-6D show that pretreatment of the decompensated CHF rats with Cialis® remarkably attenuated the adverse effects of the increased IAP on the kidney function at both the GFR (6A-6B) and RPF levels (6C-6D); and FIGS. 7A-7C show that such treatment also abolished the adverse effects of the increased IAP on the excretory functions of the kidney as evident at both the V (7A) and UNaV (7B) levels.

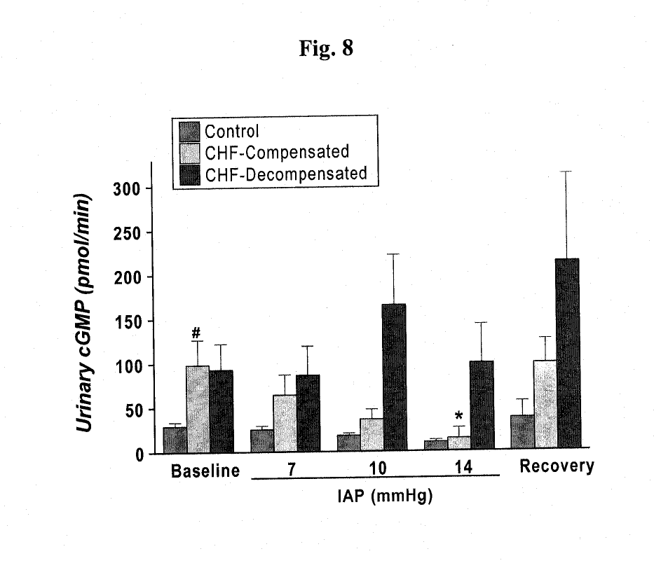

Further to its beneficial effects in preventing the adverse effects of the increased IAP on the excretory functions of the kidney as evident at both the V and UNaV levels, and on the hemodynamic effects as evident at both GFR and RPF levels, pretreatment of the decompensated CHF rats with Cialis® further prevented the inhibitory effect of IAP on urinary cGMP excretion (UcGMP V), as shown in FIG. 8.

### Example 5

### PDE5-Inhibitor Significantly Attenuates the Adverse Effects of Pneumoperitoneum on GFR, RPF, V, UnaV and UcGMP in Rats with MI

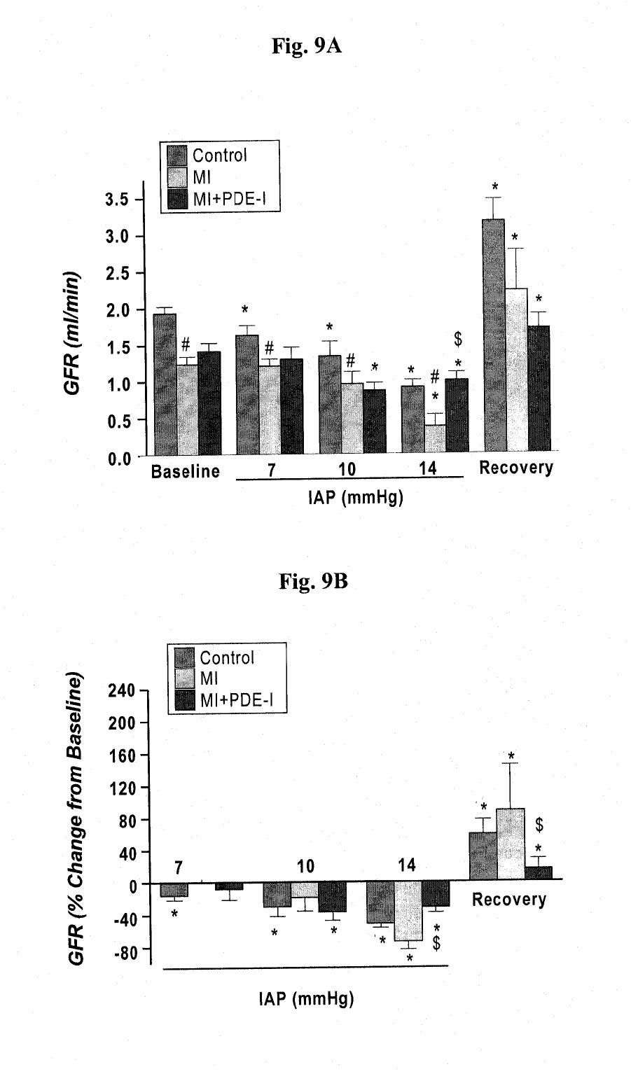

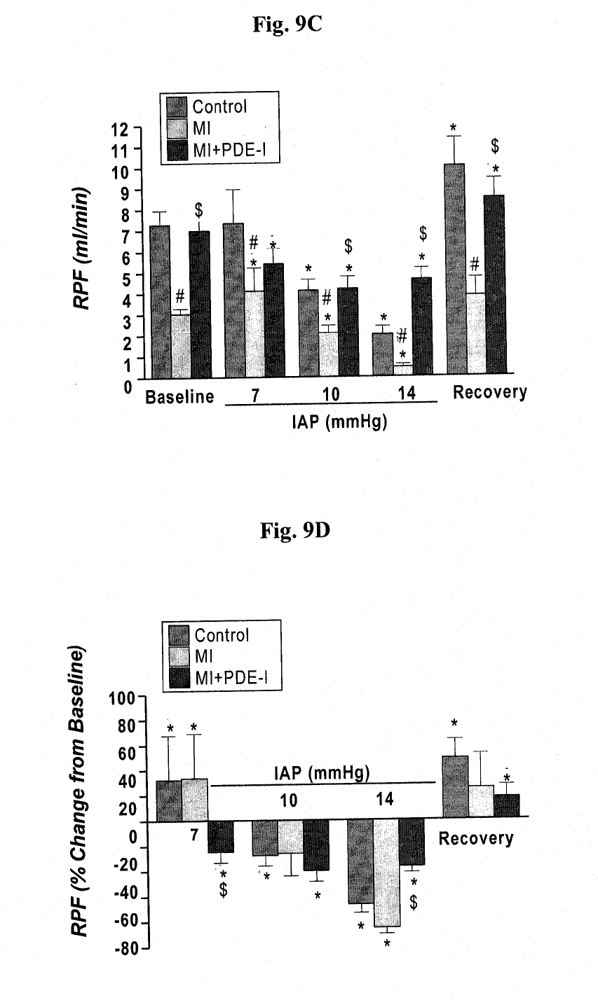

In this study we tested whether rats with heart failure induced by myocardial infarction (MI) display renal susceptibility to IAP similar to that of rats with CHF induced by the creation of ACF. As expected and shown in FIGS. 9A-9D, rats with MI had lower basal GFR and RPF compared with control animals. Increasing the IAP resulted in a decline in GFR and RPF as a function of the IAP magnitude, wherein the most adverse renal effects were noticed following the highest IAP tested, i.e., 14 mmHg. These adverse effects were more profound in rats with MI as compared with sham controls. Interestingly, when rats with MI were pretreated with Cialis®, the adverse effects of pneumoperitoneum on both GFR and RPF were substantially attenuated.

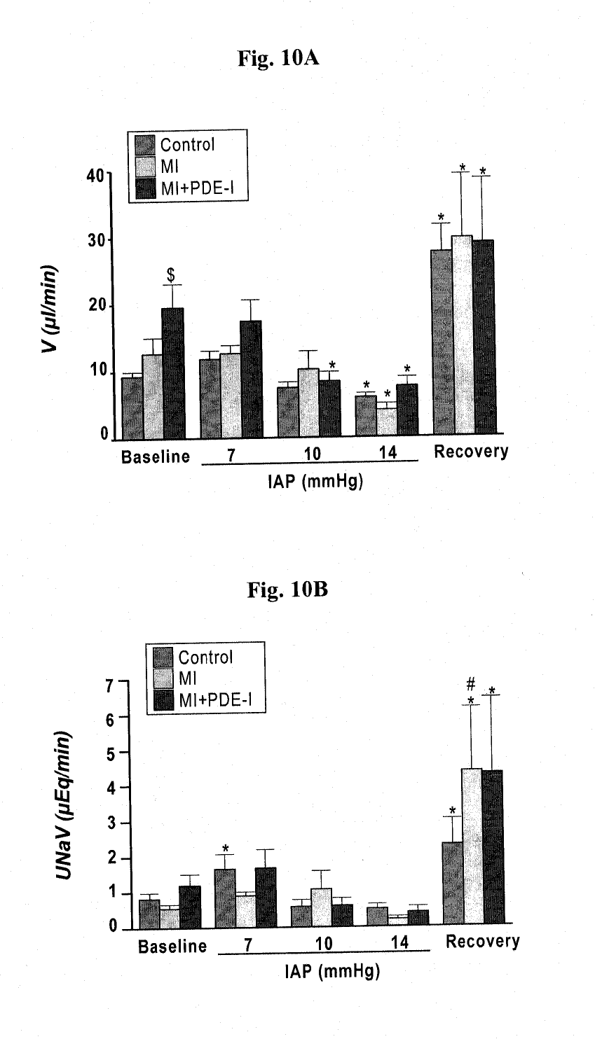

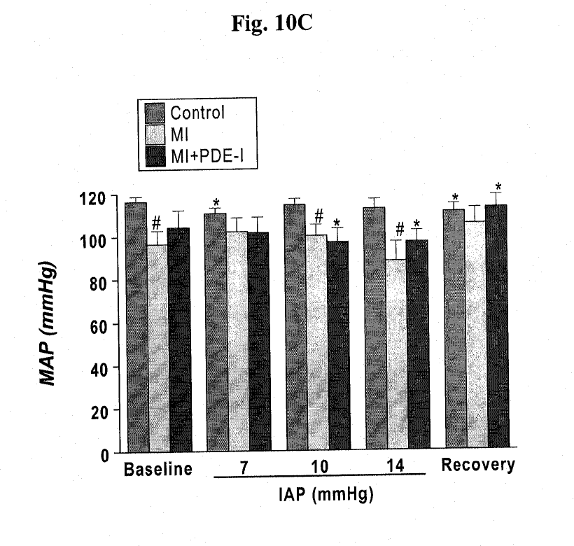

FIGS. 10A-10C show that increasing the IAP resulted in a decline in V and UnaV as a function of the IAP magnitude, wherein the most adverse renal effects were noticed following the highest IAP tested, i.e., 14 mmHg. The adverse effect of IAP on V was more profound in rats with MI as compared with sham controls. Interestingly, when rats with MI were pretreated with Cialis®, the adverse effects of pneumoperitoneum of 14 mmHg on both V and UnaV were partially ameliorated.

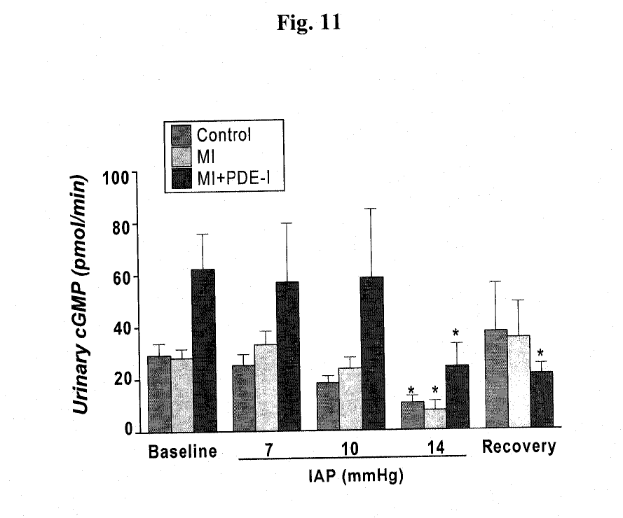

Further to its beneficial effects in preventing the adverse effects of the increased IAP on the excretory functions of the kidney as evident at both the V and UNaV levels and on the hemodynamic effects as evident at both GFR and RPF levels, pretreatment of the MI rats with Cialis® significantly attenuated the inhibitory effect of IAP on urinary cGMP excretion (UcGMP V) as well, as shown in FIG. 11.

### Example 6

### PDE5-Inhibitor Exerts Nephroprotective Effect in AKI

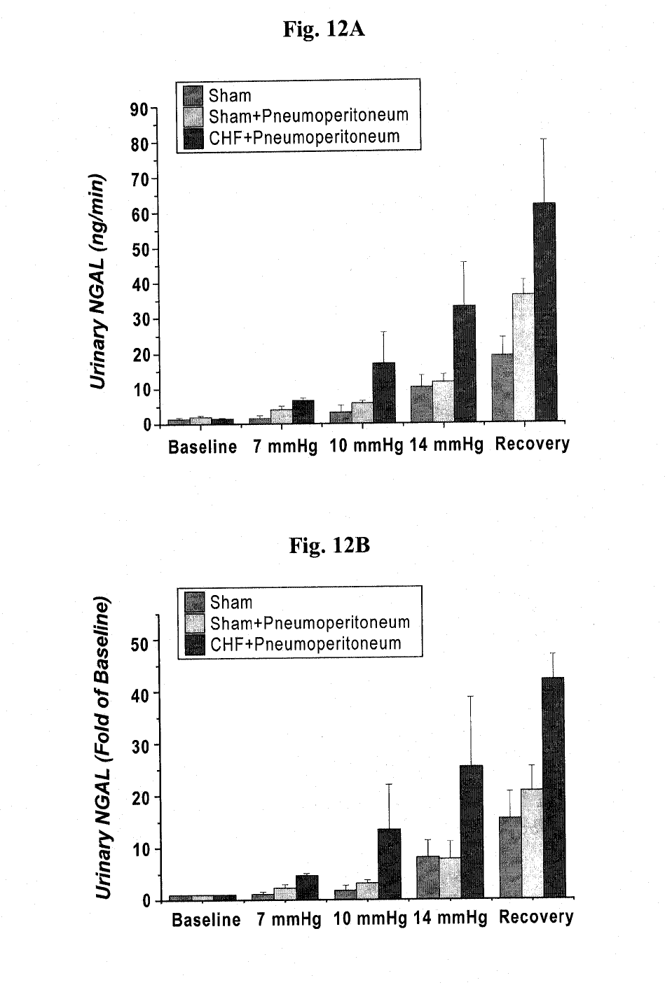

Since elevated IAP causes renal injury, in addition to renal dysfunction that is largely attributed to hemodynamic changes, an experiment was conducted so as to examine the effects of increased IAP on the urinary excretion of neutrophil gelatinase-associated lipocalcin (NGAL), a specific marker of renal injury, in normal rats and animals with congestive heart failure (CHF). Interestingly, and as shown in FIGS. 12A-12B, IAP increased in dose dependent manner the excretion of NGAL in both normal rats and rats with CHF, although the increase in the latter was much more profound than in healthy rats. These findings are in line with our observation that rats with CHF are more vulnerable to the adverse renal effects of increased IAP as expressed by severe reduction in both GFR and renal blood flow (RBF). Additionally, the stimulatory effects of IAP on excretion of NGAL provides a keen evidence that the adverse effects of IAP on the kidney function are not solely through hemodynamic changes but due to tissue injury too.

A further study was designed to explore whether warm renal ischemia for 45 min in rats (n=6) affects urinary NGAL and kidney injury molecule 1 (Kim-1) excretion. For this purpose, rats (n=6) were treated to induce renal ischemia as described in Materials and Methods, wherein rats in one group were subjected to ischemic renal injury only, and rats in the other group were pretreated with Cialis® before being subject to ischemic renal. The levels of NGAL and KIM-1 were determined in urine samples prior to acute kidney injury (AKI) and at various time points following renal artery clamping.

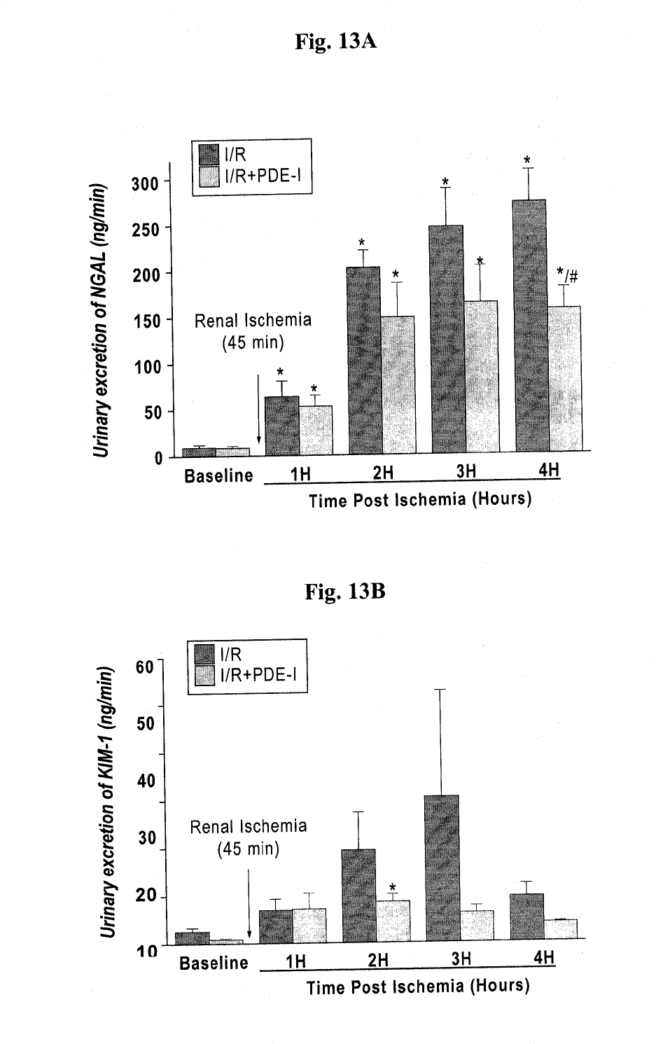

In line with our findings in human patients, experimental AKI was associated with enhanced excretion of NGAL and KIM-1 in a time dependent manner. Pretreatment with Cialis® significantly attenuated the urinary excretion of these biomarkers, suggesting that this PDE inhibitor exerts nephroprotective effect in experimental AKI (FIG. 13).

### REFERENCES

Abassi Z., Bishara B., Karram T., Khatib S., Winaver J., Hoffman A., Adverse effects of pneumoperitoneum on renal function: involvement of the endothelin and nitric oxide systems, Am J Physiol Regul Integr Comp Physiol, 2008, 294, 842-850
Ambrose J. A., Onders R. P., Stowe N. T., Simonson M. S., Robinson A. V., Wilhelm S., Schulak J. A., Pneumoperitoneum upregulates preproendothelin-1 messenger RNA, Surg Endosc, 2001, 15, 183-188
Bishara B., Karram T., Khatib S., Ramadan R., Schwartz H., Hoffman A., Abassi Z., Impact of pneumoperitoneum on renal perfusion and excretory function: beneficial effects of nitroglycerine, Surg Endosc, 2009, 23, 568-576
Borba M. R., Lopes R. I., Carmona M., Neto B. M., Nahas S. C., Pereira P. R., Effects of enalaprilat on the renin-angiotensin-aldosterone system and on renal function during CO₂ pneumoperitoneum, J Endourol., 2005, 8, 1026-1031
Chang D. T., Kirsch A. J., Sawczuk I. S., Oliguria during laparoscopic surgery, J Endourol, 1994, 8, 349-352
Chiu A. W., Azadzoi K. M., Hatzichristou D. G., Siroky M. B., Krane R. J., Babayan R. K., Effects of intra-abdominal pressure on renal tissue perfusion during laparoscopy, J Endourol, 1994, 8, 99-103
Chiu A. W., Chang L. S., Birkett D. H., Babayan R. K., A porcine model for renal hemodynamic study during laparoscopy, J Surg Res, 1996, 60, 61-68
Demyttenaere S., Feldman L. S., Fried G. M., Effect of pneumoperitoneum on renal perfusion and function: a systematic review, Surg Endosc, 2007, 21, 152-160
de Seigneux Sophie, Claude-Eric Klopfenstein, Christophe Iselin, Pierre-Yves Martin. The risk of acute kidney injury following laparoscopic surgery in a chronic kidney disease patient, Nephrol Dial Transp., 2011, 4, 339-341
Gudmundsson F. F., Viste A., Myking O. L., Bostad L., Grong K., Svanes K., Role of angiotensin II under prolonged increased intraabdominal pressure (IAP) in pigs, Surg Endosc, 2003, 17, 1092-1097
Hamilton B. D., Chow G. K., Inman S. R., Stowe N. T., Winfield H. N., Increased intra-abdominal pressure during pneumoperitoneum stimulates endothelin release in a canine model, J Endourol, 1998, 12, 193-197
Harman P. K., Kron I. L., McLachlan H. D., Freedlender A. E., Nolan S. P., Elevated intra-abdominal pressure and renal function, Ann Surg, 1982, 196, 594-597
Hazebroek E. J., de Bruin R. W., Bouvy N. D., Marquet R. L., Bonthuis F., Bajema I. M., Hayes D. P., Ijermans J. N., Bonjer H. J., Long-term impact of pneumoperitoneum used for laparoscopic donor nephrectomy on renal function and histomorphology in donor and recipient rats, Ann Surg, 2003, 237, 351-357
Ho H. S., Saunders C. J., Gunther R. A., Wolfe B. M., Effector of hemodynamics during laparoscopy: CO2 absorption or intra-abdominal pressure?, J Surg Res, 1995, 59, 497-503
Joris J. L., Chiche J. D., Canivet J. L., Jacquet N. J., Legros J. J., Lamy M. L., Hemodynamic changes induced by laparoscopy and their endocrine correlates: effects of clonidine, J Am Coll Cardiol, 1998, 32, 1389-1396
Junghans T., Bohm B., Grundel K., Schwenk W., Muller J. M., Does pneumoperitoneum with different gases, body positions, and intraperitoneal pressures influence renal and hepatic blood flow?, Surgery, 1997, 121, 206-211
Kirsch A. J., Hensle T. W., Chang D. T., Kayton M. L., Olsson C. A., Sawczuk I. S., Renal effects of CO₂ insufflation: oliguria and acute renal dysfunction in a rat pneumoperitoneum model, Urology, 1994, 43, 453-459
Khoury W., Jakowlev K., Fein A., Orenstein H., Nakache R., Weinbroum A. A., Renal apoptosis following carbon dioxide pneumoperitoneum in a rat model, J Urol., 2008, 180, 1554-1558
Kone B. C., Nitric oxide synthesis in the kidney: isoforms, biosynthesis, and functions in health, Semin Nephrol, 2004, 4, 299-315
Lahera V., Salom M. G., Miranda-Guardiola F., Moncada S., Romero J. C., Effects of NG-nitro-L-arginine methyl ester on renal function and blood pressure, Am J Physiol, 1991, 261, F1033-F1037
Lindberg F., Bergqvist D., Bjorck M., Rasmussen I., Renal hemodynamics during carbon dioxide pneumoperitoneum: an experimental study in pigs, Surg Endosc, 2003, 17, 480-484
Lindstrom P., Wadstrom J., Ollerstam A., Johnsson C., Persson A. E., Effects of increased intra-abdominal pressure and volume expansion on renal function in the rat, Nephrol Dial Transplant, 2003, 18, 2269-2277
London E. T., Ho H. S., Neuhaus A. M., Wolfe B. M., Rudich S. M., Perez R. V., Effect of intravascular volume expansion on renal function during prolonged CO₂ pneumoperitoneum, Ann Surg, 2000, 231, 195-201
Mattson D. L., Roman R. J., Cowley Jr. A. W., Role of nitric oxide in renal papillary blood flow and sodium excretion, Hypertension, 1992, 19, 766-769
McDougall E. M., Monk T. G., Wolf J. S. Jr, Hicks M., Clayman R. V., Gardner S., Humphrey P. A., Sharp T., Martin K., The effect of prolonged pneumoperitoneum on renal function in an animal model, J Am Coll Surg, 1996, 182, 317-328
Moncada S., Palmer R. M., Higgs E. A., Nitric oxide: physiology, pathophysiology, and pharmacology, Pharmacol. Rev, 1991, 43, 109-142
Nguyen N. T., Perez R. V., Fleming N., Rivers R., Wolfe B. M., Effect of prolonged pneumoperitoneum on intraoperative urine output during laparoscopic gastric bypass, J Am Coll Surg, 2002, 195, 476-483
Nishio S., Takeda H., Yokoyama M., Changes in urinary output during laparoscopic adrenalectomy, BJU Int, 1999, 83, 944-947
Nogueira J. M., Cangro C. B., Fink J. C., Schweitzer E., Wiland A., Klassen D. K., Gardner J., Flowers J., Jacobs S., Cho E., Philosophe B., Bartlett S. T., Weir M. R., A comparison of recipient renal outcomes with laparoscopic versus open live donor nephrectomy, Transplantation, 1999, 67, 722-728
Shimizu T, Tanabe K, Ishida H, Toma H., Yamaguchi Y., Histopathological evaluation of 0-h biopsy specimens of donor kidney procured by laparoscopic donor nephrectomy. Clin Transplant 2004, 18 Suppl 11, 24-28
Shimizu T., Tanabe K., Miyamoto N., et al Tokumoto T., Ishida H., Tama H., Yamaguchi Y., Early and late histopathological changes in renal allografts procured by laparoscopic donor nephrectomy. Clin Transplant 2006, 20 Suppl 15, 11-15
Ratner L. E., Hiller J., Sroka M., Weber R., Sikorsky I., Montgomery R. A., Kavoussi L. R., Laparoscopic live donor nephrectomy removes disincentives to live donation, Transplant Proc., 1997, 29, 3402-3403
Richards W. O., Scovill W., Shin B., Reed W., Acute renal failure associated with increased intra-abdominal pressure, Ann Surg, 1983, 197, 183-187
Yilmaz S., Koken T., Tokyol C., Kahraman A., Akbulut G., Serteser M., Polat C., Gokce C., Gokce O., Can preconditioning reduce laparoscopy-induced tissue injury?, Surg Endosc, 2003, 17, 819-824
Zacherl J., hein E., Stangl M., Feussner H., Bock S., Mittlbock M., Erhardt W., Siewert J. R., The influence of periarterial papaverine application on intraoperative renal function and blood flow during laparoscopic donor nephrectomy in a pig model, Surg Endosc, 2003, 17, 1231-1236

1. A method for ameliorating renal dysfunction induced by renal hypoperfusion or acute kidney injury in an individual in need thereof, said method comprising administering to said individual a therapeutically effective amount of a phosphodiesterase (PDE) inhibitor or a pharmaceutically acceptable salt or solvate thereof.

2. The method of claim 1, wherein said renal hypoperfusion or acute kidney injury results from increased intra-abdominal pressure (IAP).

3. The method of claim 2, wherein said increased IAP is caused by a laparoscopic surgery.

4. The method of claim 2, wherein said increased IAP is caused by ascites.

5. The method of claim 4, wherein said ascites is caused by cirrhosis or congestive heart failure.

6. The method of claim 4, wherein said individual is a peritoneal dialysis-treated individual having an end-stage renal disease (ESRD).

7. The method of claim 1, wherein said acute kidney injury results from renal ischemic insult.

8. The method of claim 7, wherein said renal ischemic insult is caused by endothelial dysfunction.

9. The method of claim 8, wherein
(i) said endothelial dysfunction is associated with a disease, disorder or condition selected from heart failure such as congestive heart failure, myocardial ischemia or myocardial infarction, diabetes, hypertension, hyperlipidemia, atherosclerosis, obesity or renal failure; or
(ii) said endothelial dysfunction is associated with smoking or aging.

10. The method of claim 1, wherein said acute kidney injury is caused by a radiocontrast agent or a nephrotoxic drug.

11. A method for ameliorating renal dysfunction induced by increased intraabdominal pressure (IAP) in an individual in need thereof, said method comprising administering to said individual a therapeutically effective amount of a phosphodiesterase (PDE) inhibitor or a pharmaceutically acceptable salt or solvate thereof.

12. The method of claims 11, wherein said increased IAP is caused by a laparoscopic surgery.

13. The method of claim 11, wherein said increased IAP is caused by ascites.

14. The method of claim 13, wherein said ascites is caused by cirrhosis or congestive heart failure.

15. The method of claim 13, wherein said individual is a peritoneal dialysis-treated individual having an end-stage renal disease (ESRD).

16. The method of claim 1, wherein said PDE inhibitor is a cGMP-selective PDE inhibitor.

17. The method of claim 16, wherein said cGMP-selective PDE inhibitor is a PDE type 5 inhibitor such as avanafil, lodenafil, mirodenafil, sildenafil, tadalafil, vardenafil, udenafil, dipyridamole, zaprinast, icariin, or a methoxyquinazoline, preferably tadalafil.

18. A pharmaceutical composition comprising a phosphodiesterase (PDE) inhibitor or a pharmaceutically acceptable salt or solvate thereof, and a pharmaceutically acceptable carrier, for ameliorating renal dysfunction induced by renal hypoperfusion or acute kidney injury.

19. A pharmaceutical composition comprising a phosphodiesterase (PDE) inhibitor or a pharmaceutically acceptable salt or solvate thereof, and a pharmaceutically acceptable carrier, for ameliorating renal dysfunction induced by increased intra-abdominal pressure (IAP).

20. The pharmaceutical composition of claim 18, wherein said PDE inhibitor is a cGMP-selective PDE inhibitor.

21. The pharmaceutical composition of claim 20, wherein said cGMP-selective PDE inhibitor is a PDE type 5 inhibitor such as avanafil, lodenafil, mirodenafil, sildenafil, tadalafil, vardenafil, udenafil, dipyridamole, zaprinast, icariin, and a methoxyquinazoline, preferably tadalafil.

22. A phosphodiesterase inhibitor or a pharmaceutically acceptable salt or solvate thereof for use in ameliorating renal dysfunction induced by renal hypoperfusion or acute kidney injury.

23. A phosphodiesterase inhibitor or a pharmaceutically acceptable salt or solvate thereof for use in ameliorating renal dysfunction induced by increased intraabdominal pressure (IAP).

24. Use of a phosphodiesterase inhibitor or a pharmaceutically acceptable salt or solvate thereof for the preparation of a pharmaceutical composition for ameliorating renal dysfunction induced by renal hypoperfusion or acute kidney injury.

25. Use of a phosphodiesterase inhibitor or a pharmaceutically acceptable salt or solvate thereof for the preparation of a pharmaceutical composition for ameliorating renal dysfunction induced by increased intra-abdominal pressure (IAP).

26. The pharmaceutical composition of claim 19, wherein said PDE inhibitor is a cGMP-selective PDE inhibitor.

27. The pharmaceutical composition of claim 26, wherein said cGMP-selective PDE inhibitor is a PDE type 5 inhibitor such as avanafil, lodenafil, mirodenafil, sildenafil, tadalafil, vardenafil, udenafil, dipyridamole, zaprinast, icariin, and a methoxyquinazoline, preferably tadalafil.
